(19) United States
(12) Patent Application Publication
(10) Pub. No.: US 2009/0054667 A1
(43) Pub. Date: Feb. 26, 2009

(54) Transition metal catalyzed cross-coupling of 1-halo-1-haloalkene compounds

(75) Inventors: Joseph W. Guiles, Lafayette, CO (US); Jian Qiu, Longmont, CO (US); Theodore M. Tarasow, Longmont, CO (US)

Correspondence Address: SWANSON & BRATSCHUN, L.L.C., 8210 SOUTHPARK TERRACE, LITTLETON, CO, 80120, US

(73) Assignee: Replidyne, Inc., Louisville, CO (US)

(21) Appl. No.: 11/501,418

(22) Filed: Aug. 9, 2006

Related U.S. Application Data
(60) Provisional application No. 60/706,904, filed on Aug. 10, 2005.

(51) Int. Cl. C07D 333/28; C07C 43/225

(52) U.S. Cl. 549/78; 568/656

(57) ABSTRACT

Methods for introducing a 1-halo-1-haloalkene compound onto an aromatic or heteroaromatic ring are provided, including processes for the production of certain 1-halovinyl aryl or heteroaryl derivatives in which the 1-halovinyl group is either 1-fluoro or 1-chlorovinyl and the aromatic species phenyl or thiophene, the processes including coupling an arylmagnesium species with a dihalo olefin in the presence of a nickel or iron catalyst.

### RELATED APPLICATIONS

This application claims priority from U.S. Provisional Patent Application Ser. No. 60/706,904, filed Aug. 10, 2005, entitled “Transition Metal Catalyzed Cross-Coupling of 1-Halo-1-Haloalkene Compounds,” which is hereby incorporated by reference.

### FIELD OF THE INVENTION

The present invention is directed to methods for introducing a 1-haloalkene functionality onto an aromatic or heteroaromatic ring system.

### BACKGROUND OF THE INVENTION

Chlorovinyl or fluorvinyl groups constitute useful functionality that has been introduced into synthetic medicinal compounds for the design of amide bond isosteres (Allmendinger, T. et. al. Tetrahedron Lett., 1990, 31, 7297 and mechanism-based enzyme inhibitors (McCarthy, J. R. et al. Tetrahedron Lett., 1994, 35, 5177). Processes for the manufacture make use of two general strategies i) transition metal catalyzed coupling between a metallo-vinyl species and an aryl halide or ii) transition metal catalyzed coupling between a metallo-aryl species and a dihalo-olefin. Reports have appeared regarding a process for preparing a 2′-deoxy-5-(1-fluorvinyl)uridine using a Pd-catalyzed cross coupling of a vinyl stannane (e.g. McCarthy, J. R., et al. WO 9507917A1/US94/09502;) (e.g. Spector, T., et al. WO9201452). Subsequently, McCarthy et. al. have also described a Pd-catalyzed cross coupling of a vinyl boron species (Chen, C; et al. J. of Fluorine Chemistry, 2000, 101, 285-290). In another type i process Hanamoto et. al. have described a Pd-catalyzed cross coupling of a vinyl silane (Hanamato, T.; et al. J. Org. Chem. 2003, 68, 6354-6359; Hanamato, T.; et al. Chem. Comm. 1999, 2397-2398).

Type ii processes have been described using an atypical Pd-catalyzed Heck reaction of 1,1-difluoroethylene that employ high pressure and temperature for preparing a 3-(1-fluorvinyl)-indole (Heitz, W. et al. Makromol. Chem. Rapid Commun. 1991, 12, 69; Martin, A. R., et al., Heterocycles, 1996, 43, 185).

The process described herein constitutes a novel type ii process in which an arylmagnesium species is cross coupled with a dihalo olefin in the presence of a nickel or iron catalyst. The process is best described as a Kumada-Corriu cross coupling (Tamato, K.; et al. J. Am. Chem. Soc. 1972, 94, 4374-4376; Corriu, R. J. R.; et al. Chem. Comm. 1972, 144; Banno, T.; et al. J. Organometallic Chem. 2002, 653, 288-291), and has the advantage of being high yield without requiring high temperatures or pressures. Against this backdrop the present invention was developed.

### SUMMARY OF THE INVENTION

The present invention provides methods for introducing a 1-haloalkene functionality into an organic molecule comprising reacting a substituted or unsubstituted aromatic or heteroaromatic Grignard reagent with a 1,1-dihalo-alkene in the presence of a transition metal catalyst in a cross-coupling reaction to give a 1-haloalkene substituted aromatic or heteroaromatic compound.

### DETAILED DESCRIPTION OF THE INVENTION

The present invention provides a method for the introduction of 1-haloalkene functionality into organic molecules. The invention disclosed herein uses relatively inexpensive reagents and gives good to excellent yields of the desired products with simple work-up and purification procedures.

In one embodiment, the invention provides a process for the production of 2-(fluorovinyl)thiopene carboxaldehydes by reacting a thiophene Grignard reagent, in the presence of nickel or iron chloride, with a 1,1-dihaloalkene.

In one embodiment, the invention provides a method for preparing 1-chlorovinyl or 1-fluorvinyl thiophene carboxaldehyde derivative of the following formula:

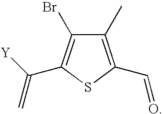

In general the present invention provides a method for introducing a 1-haloalkene functionality onto an aromatic or heteroaromatic ring by providing a Grignard reagent of the formula ArMgX, wherein Ar is a substituted or unsubstituted C₄-C₂₀ aromatic or heteroaromatic compound, and X is a halogen. Reacting ArMgX, in the presence of a transition metal catalyst M, (such as Ni, Pd, Co, Fe) with or without ligands (phosphine, nitrogen or carbene based), with a 1-halo-1-haloalkene of the formula R₁X₁X₂, wherein R₁ is a C₂-C₂₀ substituted or unsubstituted alkene including a compound of the formula (1):

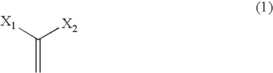

and X₁ and X₂ are halogens, and wherein X₁ and X₂ may be the same or different, in a cross-coupling reaction to give a 1-haloalkene substituted aromatic or heteroaromatic compound of the formula X₁R₁Ar. This is shown schematically as follows:

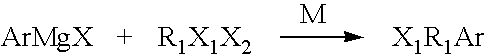

When used herein, the term “alkyl” and similar terms such as “alkoxy” includes all straight chain, branched, and cyclic isomers. Representative examples thereof include methyl, ethyl, n-propyl, iso-propyl, cyclopropyl, n-butyl, sec-butyl, iso-butyl, t-butyl, n-pentyl and n-hexyl. Optionally fluorosubstituted alkyls may have 1 or more substitutions of F for H on the alkyl chain. A representative example of an optionally fluorosubstituted alkyl is trifluoromethyl.

When used herein, the terms “alkenyl” and “alkynyl” include all straight chain, branched and cyclic isomers. Representative examples thereof include vinyl, ethynyl and 1-propynyl. Optionally fluorosubstituted alkenyls may have 1 or more substitutions of F for H on the alkenyl chain. A representative example of an optionally fluorosubstituted alkenyl is fluorovinyl.

Substituents for alkyl, alkenyl, and alkynyl groups include, for example, and unless otherwise defined, halogen, cyano, azido, nitro, carboxy, (C₁₋₆)alkoxycarbonyl, carbamoyl, mono- or di-(C₁₋₆)alkylcarbamoyl, sulpho, sulphamoyl, mono- or di-(C₁₋₆)alkylsulphamoyl, amino, mono- or di-(C₁₋₆)alkylamino, acylamino, ureido, (C₁₋₆)alkoxycarbonylamino, 2,2,2-trichloroethoxycarbonylamino, aryl, heterocyclyl, hydroxy, (C₁₋₆)alkoxy, acyloxy, oxo, acyl, 2-thienoyl, (C₁₋₆)alkylthio, (C₁₋₆)alkylsulphinyl, (C₁₋₆)alkylsulphonyl, hydroxyimino, (C₁₋₆)alkoxyimino, hydrazino, hydrazono, benzohydroximoyl, guanidino, amidino and iminoalkylamino.

In certain embodiments, “aryl” or “aromatic” refers to a mono- or bicyclic carbocyclic ring system having one or more aromatic rings including, but not limited to, phenyl, naphthyl, tetrahydronaphthyl, indanyl, indenyl and the like. When used herein, the term “heteroaryl” or “heteroaromatic” includes single or fused rings comprising one or more hetero-atoms in the ring selected from oxygen, nitrogen and sulfur. In some embodiments, the heteroaryl ring comprises from 4 to 7 ring atoms; in other embodiments, 5 to 6 ring atoms. A fused heteroaryl ring system may include carbocyclic rings and need only include one heterocyclic ring. Heteroaromatic groups can include, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. It will be appreciated that aromatic and heteroaromatic groups can be unsubstituted or substituted, wherein substitution includes replacement of one or more (e.g. 1, 2, 3 or 4) of the hydrogen atoms thereon with substituents such as are described herein or illustrated in any of the illustrative examples herein. Substituents for an aromatic or heteroaromatic group include, for example, halogen, cyano, (C₁₋₆)alkyl, mono to perfluoro(C₁₋₃)alkyl, (C₃₋₇)cycloalkyl, (C₂₋₆)alkenyl, (C₁₋₆)alkoxy, (C₂₋₆)alkenoxy, arylC₍₁₋₆₎alkoxy, halo(C₁₋₆)alkyl, hydroxy, amino, mono- or di-(C₁₋₆)alkylamino, acylamino, nitro, carboxy, (C₁₋₆)alkoxycarbonyl, (C₁₋₆)alkenyloxycarbonyl, (C₁₋₆)alkoxycarbonyl(C₁₋₆)alkyl, carboxy(C₁₋₆)alkyl, (C₁₋₆)alkylcarbonyloxy, carboxy(C₁₋₆)alkyloxy, (C₁₋₆)alkoxycarbonyl(C₁₋₆)alkoxy, (C₁₋₆)alkylthio, (C₁₋₆)alkylsulphinyl, (C₁₋₆)alkylsulphonyl, sulphamoyl, mono- and di-(C₁₋₆)-alkylsulphamoyl, carbamoyl, mono- and di-(C₁₋₆)alkylcarbamoyl, and heterocyclyl.

When used herein, the terms “halogen” and “halo” include fluorine, chlorine, bromine and iodine and fluoro, chloro, bromo and iodo, respectively, unless otherwise indicated.

In some embodiments, the substituted or unsubstituted aromatic or heteroaromatic Grignard ArMgX is formed via the reaction between a Grignard reagent of formula R₂MgX, with a substituted or unsubstituted aromatic or heteroaromatic compound Ar, wherein Ar is as previously defined, and wherein R₂ is an aliphatic hydrocarbon group, such as a C₁-C₂₀ alkyl group. In Example 1, below, Ar is 2,3-dibromo-5-dimethoxymethyl-4-methylthiophene, Grignard reagent ArMgX is 3-bromo-4-methyl-5-dimethylthiophenyl-1-magnesium chloride, R₁X₁X₂ is 1-bromo-1-fluoroethylene, and the product X₁R₁Ar is 3-bromo-5-dimethoxymethyl-2-(1-fluorovinly)-4-methylthiophene. In Example 3, Grignard reagent is 3-bromo-5-dimethoxymethyl-4-methylthiophenyl-1-magnesium chloride 2, R₁X₁X₂ is 1,1-difluoroethylene, and the product X₁R₁Ar is 3-bromo-5-dimethoxymethyl-2-(1-fluorovinly)-4-methylthiophene. Additional specific examples are shown throughout the Examples section.

In some embodiments, the invention provides methods for the introduction of 1-fluoroethylene functionality into organic molecules. Specifically, the cross coupling between 1-halo(bromo, chloro or fluoro)-1-fluoroethylene and an aryl or heteroaryl Grignard reagent is catalyzed by a transition metal catalyst (such as Ni, Pd, Co, Fe) with or without ligands (phosphine, nitrogen or carbene based).

In some embodiments, the cross coupling between 1-bromo-1-fluoroethylene or 1-chloro-1-fluoroethylene and an aryl or heteroaryl Grignard reagent is catalyzed by Ni(0)/Ni(II) species.

It will be appreciated that certain compounds produced by the methods of the invention may comprise one or more chiral centers so that compounds produced may exist as stereoisomers, including diastereoisomers and enantiomers, and mixtures thereof, including racemates.

In one embodiment, the invention provides a method for introducing a 1-haloalkene functionality into an organic molecule comprising providing an aromatic or heteroaromatic Grignard reagent, reacting the substituted or unsubstituted aromatic or heteroaromatic Grignard reagent with a 1-halo-1-haloalkene and a transition metal catalyst in a cross-coupling reaction to give a 1-haloalkene substituted aromatic or heteroaromatic compound.

In some embodiments the method further comprises isolating the 1-haloalkene substituted aromatic or heteroaromatic compound.

The 1-halo-1-haloalkene may be, for example, 1-bromo-1-fluoroethylene, 1,1-difluoroethylene, 1-chloro-1-fluoroethylene, and 1,1-dichloroethylene.

Any transition metal catalyst can be used in this invention, such as Ni, Pd, Co, Fe, Ru, and Pt. The transition metal catalyst can be used with or without ligands. Any suitable ligand can be used in the invention such as phosphine, nitrogen, or carbene ligands, for example Ph₃P, BINAP, 2-(Di-t-butylphosphino)biphenyl, DPPF and nucleophilic carbenes (1,3-bis(2,6-di-1-propylphenyl)imidazolium chloride and 1,3-bis(2,4,6-trimethylphenyl)imidazolium.

The methods of the present invention can include isolation of the compounds produced or purification of the compounds produced by conventional methods. These methods can include, for example, filtration, recrystallization, solvent extraction, distillation, precipitation, sublimation, column chromatography and the like, as is well known to those skilled in the art. The products may be analyzed and/or checked for purity by conventional methods such as, for example, thin layer chromatography, NMR, HPLC, melting point, mass spectral analysis, elemental analysis and the like, well known to those skilled in the art. The compounds according to the invention are suitably isolated/purified to at least 50% pure, suitably at least 60% pure, advantageously at least 75% pure, preferably at least 85% pure and more preferably at least 95% pure. All percentages are calculated as weight/weight. Purity is determined against unreacted species, by products, and other compounds found in association with the product.

An impure or less pure form of a compound according to the invention may, for example, be used in the preparation of a more pure form of the same compound.

For example, in Example 1, 3-bromo-5-dimethoxymethyl-2-(1-fluorovinyl)-4-methylthiophene is hydrolyzed with aqueous acetic acid to prepare 4-bromo-5-(1-fluorovinyl)-3-methyl-2-thiophenecarboxaldehyde.

### EXAMPLES

The following examples are provided for illustrative purposes only and are not intended to limit the scope of the invention.

### Example 1

### Preparation of 3-bromo-5-dimethoxymethyl-2-(1-fluorovinyl)-4-methylthiophene 3 and 4-bromo-5-(1-fluorovinyl)-3-methyl-2-thiophenecarboxaldehyde 4

Ethylmagnesium chloride (2 M in THF, 10 mL, 20 mmol) was added over 5 minutes to a suspended solution of 2,3-dibromo-5-dimethoxymethyl-4-methylthiophene (1) (5.10 g, 15.4 mmol) in anhydrous, deoxygenated toluene (10 mL) under an argon atmosphere, cooled with an ice/water bath (0-5° C.). After the addition of EtMgCl, the resultant clear dark solution was stirred at room temperature for 1 h. (Christophersen, C.; et al. J. Org. Chem. 2003, 68, 9513-9516). To the solution, 1-bromo-1-fluoroethylene (2.9 g, 23.2 mmol, 1.5 equiv.) in anhydrous, deoxygenated toluene (4.0 mL) was added, chilled with an ice/NaCl bath (−15° C.). Then, nickel (II) chloride (40 mg, 0.31 mmol) was added immediately. The mixture was stirred for 6 h while slowly warming to ambient temperature. The reaction was quenched with water (0.5 mL) followed by adding 30% ethylacetate/hexane (60 mL) and alumina (12 g, activated basic aluminum oxide, Brockmann I, standard grade, approximate 150 mesh). After stirring for 20 min, filtration and concentration under reduced pressure afforded 3-bromo-2-(1-fluoroethenyl)-5-dimethoxymethyl-4-methylthiophene (3) as a dark oil (4.35 g, 95%). ¹H NMR: 5.62 (1H, s), 5.40 (1H, dd, J=4.0, 50 Hz), 5.0 (1H, dd, J=4.0, 18.4 Hz), 3.33 (6H, s), 2.22 (3H, s) ppm. ¹⁹F NMR: −92 (dd, J=18, 50 Hz) ppm; MS (ES+) 265/263 (M⁺-OMe).

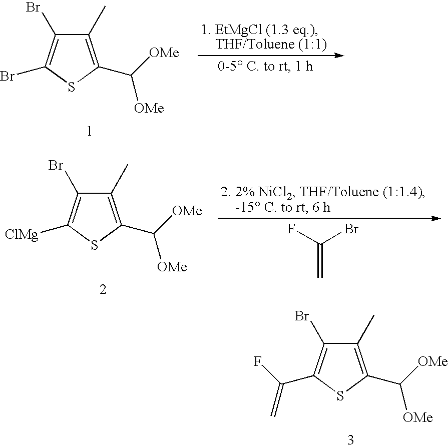

The crude 3-bromo-5-dimethoxymethyl-2-(1-fluoroethenyl)-4-methylthiophene 3 (4.35 g) was dissolved in acetic acid (16 mL). To the above solution, water (4.0 mL) was added. After stirring for 2 h, some yellow precipitates formed. The product was precipitated out by adding water (40 mL). Filtration and drying under vacuum afforded a yellow solid (3.45 g, 90%), which was recrystallized from hexane to afford 4-bromo-5-(1′-fluorovinyl)-3-methyl 2-carboxaldehydethiophene (4) as a pale yellow solid (3.0 g, 80%). ¹H NMR: 10.05 (1H, s), 5.70 (1H, ddd, J=1.2, 4.0, 50 Hz), 5.0 (1H, ddd, J=0.8, 4.0, 18.4 Hz), 2.57 (3H, s) ppm. ¹⁹F NMR: −92.20 (dd, J=18, 50 Hz) ppm; MS (ES+) 251/249 (M+H⁺).

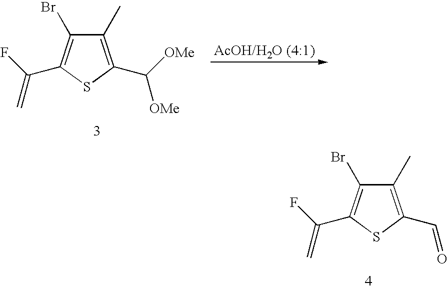

### Example 2

### The Scale-up preparation of 3-bromo-5-dimethoxymethyl-2-(1-fluoroethenyl)-4-methylthiophene 3

To a 12-L, four necked, round bottom flask equipped with a mechanical stirrer, nitrogen inlet, thermometer and addition funnel was added 2,3-dibromo-5-dimethoxymethyl-4-methylthiophene 1 ((200.0 g, 606.0 mmol), followed by 1.6 L of anhydrous toluene under a nitrogen atmosphere. The solution was cooled to 5° C. with an ice-bath and ethylmagnesium chloride (365 mL, 730 mmol, 2M in THF) added while maintaining a reaction temperature below 10° C. After complete addition, the reaction was stirred in the ice-bath for 2.5 h. Nickel chloride (1.50 g, 11.6 mmol) was then added followed by 1-bromo-1-fluoroethylene (106.0 g, 848.4 mmol). The mixture was gradually warmed to room temperature overnight.

The reaction was quenched with the addition of water (34.0 g, 1888.9 mmol), followed by the addition of 30% ethyl acetate/heptane (1.2 L). To this mixture was then added basic alumina (185.0 g, 1814.4 mmol). The mixture was stirred for 25 min., and filtered through a coarse glass fritted funnel. The filter cake was washed with 30% ethyl acetate/heptane (800 ml). The filtrate was concentrated under reduced pressure on a rotary evaporator to afford the desired product 3 as a dark brown oil (212.0 g, contaminated with toluene).

### Example 3

### Preparation of 3-bromo-5-dimethoxymethyl-2-(1-fluorovinyl)-4-methylthiophene 3 by using 1,1-difluoroethylene

1,1-Difluoroethylene (1.8 g, 28 mmol) was condensed in a sealable pressure tube, containing dry and degassed toluene (1.5 mL), with a liquid nitrogen cooling bath. Then, nickel (II) chloride (4.8 mg) and 1,3-bis(2,6-di-i-propylphenyl)imidazolium chloride (16.2 mg) were introduced under an argon atmosphere. The mixture was stirred at room temperature for one hour. The resultant solution was cooled by a liquid nitrogen bath and a solution of the Grignard reagent 2 [0.63 mmol, 1.5 mL, 0.42 M, pre-made from 2,3-dibromo-5-dimethoxymethyl-4-methylthiophene 1 (1.26 g) in dry, degassed toluene (6 mL) and ethylmagnesium chloride (2.7 mmol)] was introduced under an argon atmosphere. The reaction mixture was stirred for 64 h at room temperature. The pressurized reaction vessel was chilled with a liquid nitrogen bath, released to the air and slowly warmed to room temperature. The reaction mixture was diluted with 60 mL of ether/hexane (1:1) and passed through a pad of neutral Alumina (pH: 6.8), washing with 1:1 ether/hexane. Removal of solvent under reduced pressure afforded 3-bromo-2-(1-fluoroethenyl)-5-dimethoxymethyl-4-methylthiophene 3 as a dark oil (180 mg, 80% pure, yield: 75%).

### Example 4

### Preparation of 3-bromo-5-dimethoxymethyl-2-(1-fluorovinyl)-3-methylthiophene 3 by using 1-chloro-1-fluoroethlyene

Following the same process in example 1, 2,3-dibromo-5-dimethoxymethyl-4-methylthiophene 1 (2.23 g, 6.71 mmol) in dry, degassed toluene (8.0 mL) was treated with ethylmagnesium chloride (4.35 mL, 8.7 mmol, 2M in THF). The resultant Grignard solution 2 was mixed with a solution of 1-chloro-1-fluoroethylene (1.2 g, 14.9 mmol) in toluene (3.0 mL) and nickel chloride (26 mg, 0.201 mmol) in a sealable pressure tube under an argon atmosphere, cooled with a dry ice/acetone bath. The reaction solution was stirred at room temperature overnight and was quenched by adding water (0.5 mL), and further diluted with ether/hexane (1:1, 60 mL). The resultant suspended solution was passed through a pad of Alumina (pH 6.8) and the pad was washed with ether. Removal of organic solvents afforded the crude product 3 as dark oil (1.4 g, yield: 70%).

### Example 5

### Preparation of 4-bromo-2-dimethoxymethyl-5-(1-fluorovinyl)-3-methylthiophene 3 by using FeCl₂

Following the same process in Example 1, 2,3-dibromo-5-dimethoxymethyl-4-methylthiophene 1 (1.0 g, 2.03 mmol) in dry, degassed toluene (4.0 mL) was treated with ethylmagnesium chloride (4.0 mmol, 2M in THF). The resultant Grignard solution 2 was reacted with a solution of 1-bromo-1-fluoroethylene (0.56 g, 4.54 mmol) in toluene (5.0 mL) and iron (II) chloride (19.2 mg, 0.151 mmol). The reaction solution was stirred at room temperature overnight and was quenched by adding water (0.5 ml), and further diluted with ether/hexane (1:1, 60 mL). The resultant suspended solution was passed through a pad of Alumina (pH 6.8) and the pad was washed with ether. Removal of organic solvents afforded the desired crude product 3 as dark oil (0.24 g, yield: 40%).

### Example 6

### Preparation of 4-bromo-5-(1-chlorovinyl)-2-dimethoxymethyl-3-methylthiophene 5

Following the same process in the example 1, the Grignard solution 2 (2.0 mL, 1.2 mmol) in toluene/THF (1:1) was treated with nickel (II) chloride (7.7 mg, 0.06 mmol) and 1,1-dichloroethylene (1.2 g, 12 mmol) at room temperature for 16 h. A dark oil 5 (280 mg, 75%) was afforded. ¹H NMR: 6.05 (1H, s), 5.82 (1H, s), 5.80 (1H, s), 3.28 (6H, s), 2.22 (3H, s) ppm. MS (ES) 281 (M⁺-OMe) and 279 (M⁺-OMe).

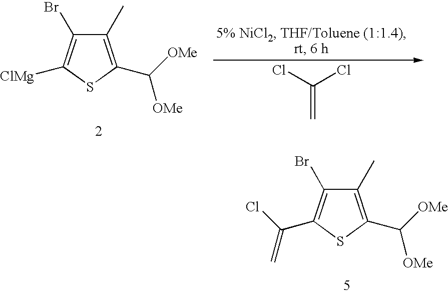

### Example 7

### Preparation of methyl 4-(1-fluorovinyl)benzoate (7)

Methyl 4-iodobenzoate 6 (0.39 g, 1.49 mmol) in THF (3.0 ml) was carefully treated with isopropyl magnesium chloride (0.82 mL, 2.0 M in THF) under an argon atmosphere with a NaCl/ice bath (−20° C.) for 2 h. Then the resultant Grignard solution was introduced a sealable pressure tube, which contained 1-bromo-1-fluoroethylene (0.28 g, 2.23 mmol) in THF (1.0 ml) and 1,3-bis(diphenylphosphino)propane nickel (II) chloride (40 mg, 0.074 mmol), chilled with a NaCl/ice water bath. The resultant solution was stirred for 3 h, quenched with 0.5 ml of water and diluted with ether/hexane (1:3, 25 mL). The organic solution was filtered through a pad of Alumina, washing with ether. Removal of solvents afforded a mixture. The mixture was purified by a silica gel column chromatograph to give methyl 4-(1-fluoro-vinyl)benzoate 7 as a pure colorless solid (188 mg, 70%). ¹H NMR: 8.04 (1H, d, J=8.0 Hz), 7.61 (1H, d, 8.0 Hz), 5.2 (1H, dd, J=3.2, 49 Hz), 4.98 (1H, dd, J=3.2, 17 Hz), 3.93 (1H, s) ppm; ¹⁹F NMR: −108.6 (dd, J=49, 17 Hz) ppm.

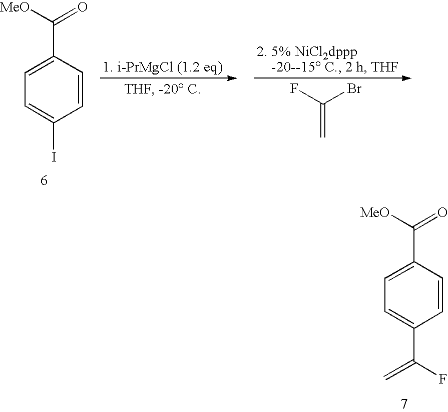

### Example 8

### Preparation of 5-bromo-1-dimethoxymethyl-2-ethoxy-3-(1-fluorovinyl)benzene (9) and 5-bromo-2-ethoxy-3-(1-fluorovinyl)benzaldehyde (10)

In a sealable pressure tube, ethylmagnesium chloride (0.179 mL, 2M in THF, 0.358 mmole) was added to a solution of 5-bromo-1-dimethoxymethyl-2-ethoxy-3-iodo-benzene (8) (107 mg, 0.276 mmole) in anhydrous, deoxygenated toluene (1.0 mL) under an argon atmosphere at ambient temperature. The resultant solution was stirred at ambient temperature for 1 h and then cooled to −5° C. and treated with nickel chloride (3.6 mg, 0.028 mmol). The argon atmosphere was then exchanged with 1-bromo-1-fluoroethylene (˜300 mg) and the reaction was allowed to come slowly to ambient temperature and stirred for 48 h. Ether/hexane (1:1, 10 mL) was then added to the reaction mixture and the resultant suspension was filtered through a pad of Alumina and the reaction vessel and alumina washed with 1:1 ether/hexane (4×10 mL). The combined filtrates were concentrated under reduced pressure to give crude 9 as clear oil. The acetal protecting group was removed by dissolving the crude product in CHCl₃ (3 mL) and stirring with 1:1 TFA/H₂O (1 mL) at ambient temperature for 4 hours. The reaction mixture was neutralized with Na₂CO₃ (15 mL) and extracted with CH₂Cl₂ (3×30 mL). The combined organic extracts were dried over MgSO₄ and the solvent removed under reduced pressure. Purification by flash silica gel chromatography gave the desired product 10 (43 mg) as a white solid. ¹H NMR: 10.33 (1H, s), 7.95 (1H, d J=2.4 Hz), 7.86 (1H, d, J=2.4 Hz), 5.40 (1H, dd, J=2.4, 51 Hz), 5.18 (1H, dd, J=3.2, 19 Hz), 4.07 (2H, quart., J=7.0 Hz), 1.44 (3H, t, J=7.0 Hz) ppm; ¹⁹F NMR: −99.80 (dd, J=18, 51 Hz).

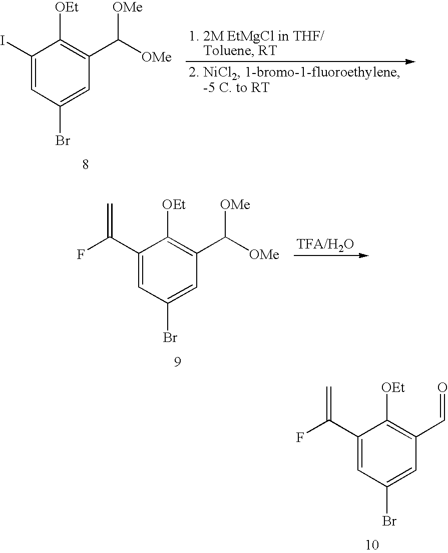

What is claimed is:

1. A method of introducing a 1-haloalkene functionality into an organic molecule comprising:
a) providing a substituted or unsubstituted aromatic or heteroaromatic Grignard reagent of the formula ArMgX, wherein Ar is a substituted or unsubstituted C₄-C₂₀ aromatic or heteroaromatic compound, and X is a halogen; and
b) reacting the substituted or unsubstituted aromatic or heteroaromatic Grignard reagent with a 1-halo-1-haloalkene of the formula R₁X₁X₂, wherein R₁ is an C₂-C₂₀ substituted or unsubstituted alkene and X₁ and X₂ are halogens, in the presence of a transition metal catalyst M, to give a 1-haloalkene substituted aromatic or heteroaromatic compound of the formula X₁R₁Ar.

2. The method of claim 1, further comprising isolating the 1-haloalkene substituted aromatic or heteroaromatic compound.

3. The method of claim 1 wherein X, and X₂ are different halogens

4. The method of claim 1 wherein X, and X₂ are the same halogen.

5. The method of claim 1, wherein the transition metal catalyst is selected from the group consisting of Ni, Pd, Co, Fe.

6. The method of claim 1 wherein the metal catalyst M further comprises a ligand.

7. The method of claim 6 wherein the ligand is selected from the group consisting of phosphine, nitrogen and carbine.

8. The method of claim 1, wherein the substituted or unsubstituted aromatic or heteroaromatic Grignard reagent is formed by reacting a Grignard reagent of the formula R₂MgX, wherein R₂ is a C₁-C₂₀ alkyl group, with a substituted or unsubstituted aromatic or heteroaromatic compound.

9. The method of claim 1, wherein the 1-halo-1-haloalkene is selected from the group consisting of 1-bromo-1-fluoroethylene, 1-chloro-1-fluoroethylene, 1,1-dichloroethylene and 1,1-difluoroethylene.

10. A method for preparing 4-bromo-2-dimethoxymethyl-5-(1-fluorovinyl)-3-methylthiophene comprising:
a) reacting with a Grignard reagent of the formula RMgX to form 3-bromo-5-dimethoxymethyl-4-methylthiophenyl magnesium chloride; and
b) reacting the 3-bromo-5-dimethoxymethyl-4-methylthiophenyl magnesium chloride with a compound selected from the group consisting of 1-bromo-1-fluoro-etheylene, 1-chloro-1-fluoroethylene, 1,1-difluoroethylene, wherein the transition metal catalyst M is NiCl₂ or FeCl₂ to form 3-bromo-5-dimethoxymethyl-2-(1-fluorovinyl)-4-methylthiophene.

11. A method of preparing 4-bromo-5-(1-fluorovinyl)-3-methyl-2-thiophene carboxaldehyde comprising:
a) preparing 3-bromo-5-dimethoxymethyl-2-(1-fluorovinyl)-4-methylthiophene by the method of claim 10; and
b) hydrolyzing the 4-bromo-2-dimethoxymethyl-5-(1-fluorovinyl)-3-methylthiophene,
whereby 4-bromo-5-(1-fluorovinyl)-3-methyl-2-thiophene carboxaldehyde is prepared.

12. The method of claim 11, wherein the hydrolysis is performed by contacting the 4-bromo-2-dimethoxymethyl-5-(1-fluorovinyl)-3-methylthiophene with an aqueous acid solution.

13. A method for preparing 4-bromo-2-dimethoxymethyl-5-(1-chlorovinyl)-3-methylthiophene comprising:
a) reacting with a Grignard reagent of the formula RMgX to form 3-bromo-5-dimethoxymethyl-4-methylthiophenyl magnesium chloride; and
b) reacting the 3-bromo-5-dimethoxymethyl-4-methylthiophenyl magnesium chloride with a compound selected from the group consisting of 1,1-dichloroethylene wherein the transition metal catalyst M is NiCl₂ or FeCl₂ to form 3-bromo-5-dimethoxymethyl-2-(1-chlorovinyl)-4-methylthiophene.

14. A method of preparing 4-bromo-5-(1-chlorovinyl)-3-methyl-2-thiophene carboxaldehyde comprising:
a) preparing 3-bromo-5-dimethoxymethyl-2-(1-fluorovinyl)-4-methylthiophene by the method of claim 10; and
b) hydrolyzing the 4-bromo-2-dimethoxymethyl-5-(1-chlorovinyl)-3-methylthiophene,
whereby 4-bromo-5-(1-chlorovinyl)-3-methyl-2-thiophene carboxaldehyde is prepared.

15. The method of claim 14, wherein the hydrolysis is performed by contacting the 4-bromo-2-dimethoxymethyl-5-(1-chlorovinyl)-3-methylthiophene with aqueous acetic acid.
